(19) United States
(12) Patent Application Publication
(10) Pub. No.: US 2011/0137384 A1
(43) Pub. Date: Jun. 9, 2011

(54) Far Infrared Waist Belt

(76) Inventor: Shyan-Wei Chen, Linkou 2nd Industrial Park (TW)

(21) Appl. No.: 12/631,589

(22) Filed: Dec. 4, 2009

(51) Int. Cl. A61N 5/06; D03D 3/00; A41F 9/00

(52) U.S. Cl. 607/88; 139/384 R; 2/322

(57) ABSTRACT

A far infrared waist belt of which the belt body has multiple woven zones connected one after another, each formed of multiple weft threads woven with spandex warp yarns and non-stretchable warp yarns and far infrared radiating warp threads containing a negatively ionized nanometered far infrared radiating substance and far infrared radiating warp stitch thread containing a negatively ionized nanometered far infrared radiating substance in a crossed manner to provide a high structural strength and to radiate far infrared rays for stimulating the user's blood circulation.

### BACKGROUND AND SUMMARY OF THE INVENTION

The present invention relates to a waist belt for fastening to a person's waist and more particularly, to a far infrared waist belt that radiates far infrared rays to stimulate the user's blood circulation and that contains negative ions to provide a sterilizing effect.

A regular waist belt is simply a part of clothing worn around the waist to hold trousers up or as a fashion accessory. There are waist belt manufacturers who have their waist belt products printed or coated with a layer of far infrared radiating substance to radiate far infrared rays. However, the printed or coated with a layer of far infrared radiating substance may drop from the body of the waist belt or lose their function quickly with the use of the waist belt.

Taiwan Patent Number I277429, entitled “Nanometered far infrared patch”, teaches the use of a patch that radiates far infrared rays. This design is not suitable for wearing on the body of a person.

The present invention has been accomplished under the circumstances in view. It is therefore the main object of the present invention to provide a far infrared waist belt that radiates far infrared rays. It is another object of the present invention to provide a far infrared waist belt, which is durable in use.

According to one aspect of the present invention, the far infrared waist belt comprises a belt body, a buckle fastened to one end of the belt body, and a tailpiece fastened to the opposite end of the belt body. The belt body comprises multiple woven zones connected one after another. Each woven zone is formed of multiple weft threads woven with spandex warp yarns and non-stretchable warp yarns and far infrared radiating warp threads containing a negatively ionized nanometered far infrared radiating substance and far infrared radiating warp stitch thread containing a negatively ionized nanometered far infrared radiating substance in a crossed manner. Therefore, the belt body has a high structural strength and radiates far infrared rays to stimulate the user's blood circulation.

### BRIEF DESCRIPTION OF THE DRAWING

FIG. 1 is an elevational view of a far infrared waist belt in accordance with the present invention.

FIG. 2 is a plain view in an enlarged scale of a part of the belt body of the far infrared waist belt in accordance with the present invention.

FIG. 3 is a woven pattern table showing the arrangement relationship of the weft and warp threads and yarns of the front edge zone of the belt body of the far infrared waist belt in accordance with the present invention.

FIG. 4 is a woven pattern table showing the arrangement relationship of the weft and warp threads and yarns of each woven zone of the belt body of the far infrared waist belt in accordance with the present invention.

FIG. 5 is a woven pattern table showing the arrangement relationship of the weft and warp threads and yarns of the rear selvedge zone of the belt body of the far infrared waist belt in accordance with the present invention.

### DETAILED DESCRIPTION OF THE INVENTION

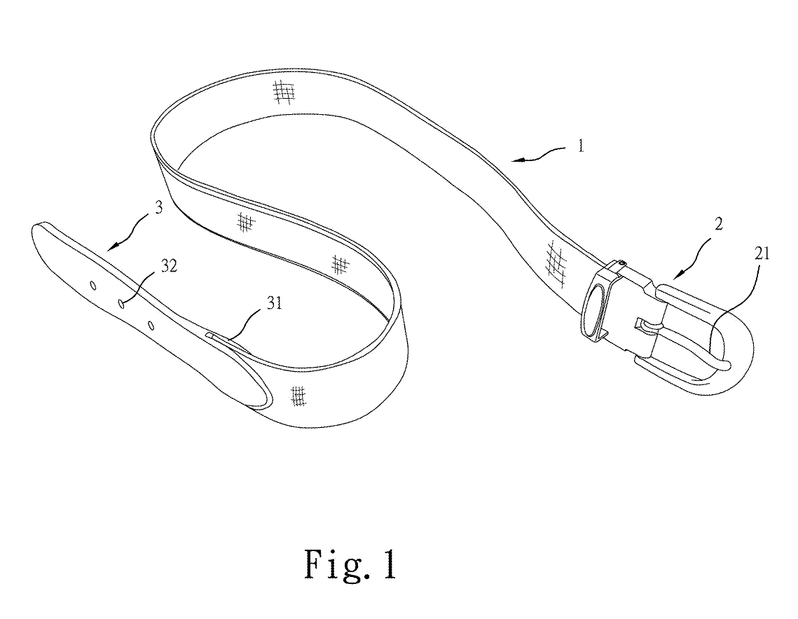

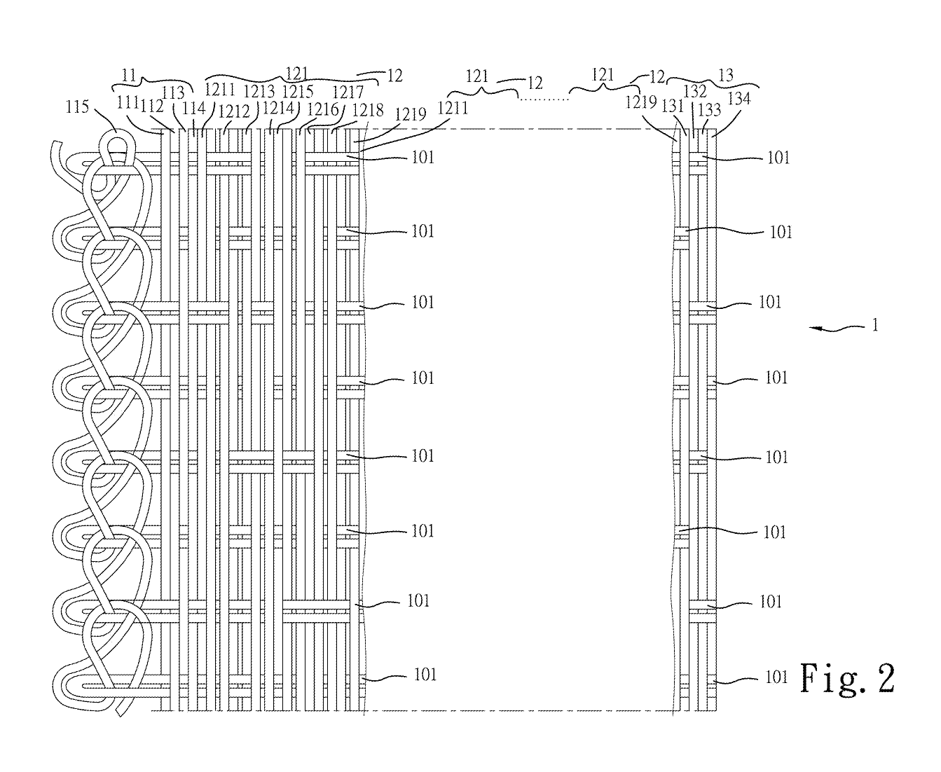

Referring to FIGS. 1 and 2, a far infrared waist belt in accordance with the present invention is shown comprising a belt body 1, a buckle 2 fastened to the front end of the belt body 1 and a tailpiece 3 affixed to the rear end of the belt body 1. The tailpiece 3 has a series of punch holes (or eyelets) 32 cut through its top and bottom sides and arranged along its length. The buckle 2 has a prong 21 for selectively inserting through one of the punch holes 32 to join the ends of the far infrared waist belt.

The main features of the far infrared waist belt are outlined hereinafter. The belt body 1 can be made of an elastic material. Alternatively, the belt body 1 can be made of a non-elastic material. Further, the belt body 1 comprises a front edge zone 11 and multiple woven zones 12. The first woven zone 12 is joined to the front edge zone 11.

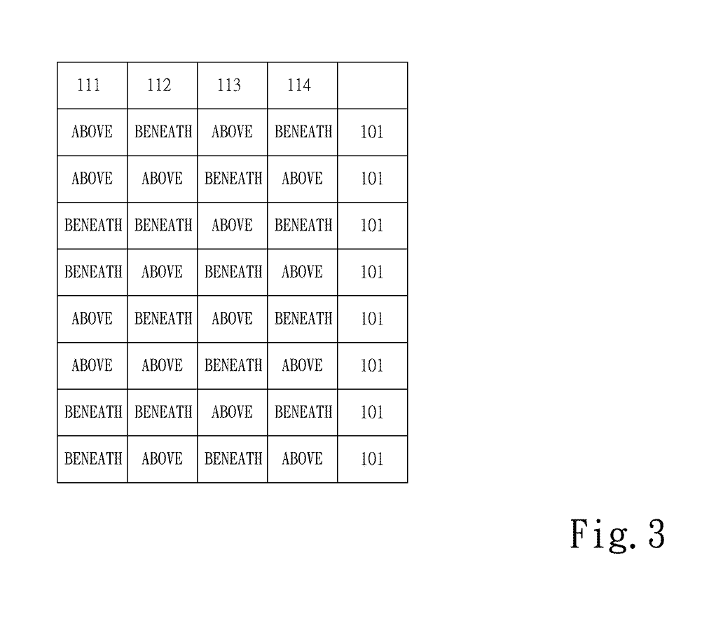

The front edge zone 11 comprises multiple spandex warp yarns or filament yarns 111 and 112, and multiple far infrared radiating warp threads 113 and 114 containing a negatively ionized nanometered far infrared radiating substance, multiple weft threads 101 and a selvedge thread 115. The spandex warp yarns or filament yarns 111 and 112 and the far infrared radiating warp threads 113 and 114 are woven with the weft threads 101 subject to the pattern shown in FIG. 3. For example, the spandex warp yarn or filament yarn 111 extend over the top side of the first and second weft threads 101, and then beneath the third and fourth weft threads 101, and then over the top side of the fifth and sixth weft threads 101 and then beneath the seventh and eighth weft threads 101; the far infrared radiating warp thread 114 extends beneath the first weft thread 101, and then over the top side of the second weft thread 101, and then beneath the third weft thread 101, and then over the top side of the fourth weft thread 101, and then beneath the fifth weft thread 101, and then over the top side of the sixth weft thread 101, and then beneath the seventh weft thread 101 and finally over the top side of the eighth weft thread 101. The selvedge thread 115 is knitted on the weft thread 101 at the front side of the belt body 1.

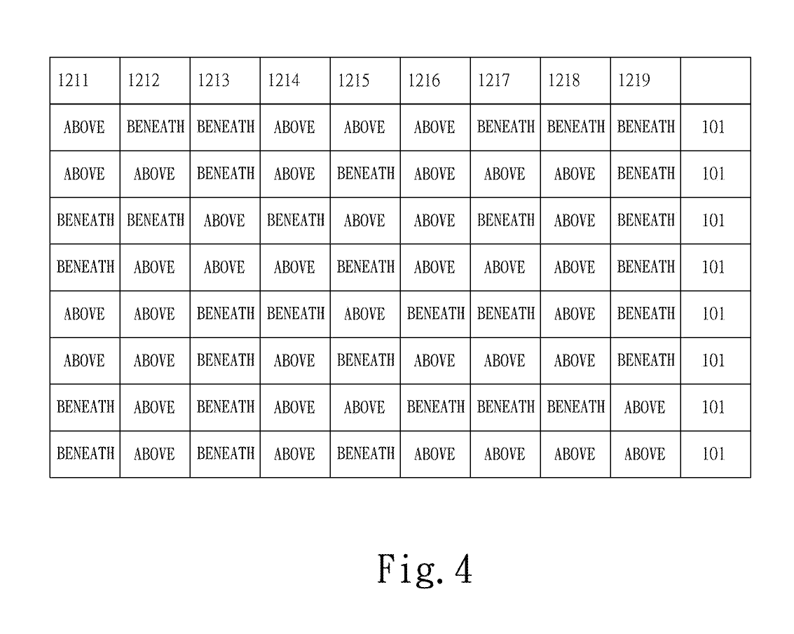

Each woven zone 12 is comprised of multiple warp thread units 121 woven with multiple weft threads 101 in a crossed manner. Each warp thread unit 121 comprises a front spandex warp yarn or filament yarn 1211, multiple non-stretchable warp yarns 1212, 1214, 1216 and 1218, multiple far infrared radiating warp threads 1213 and 1219 containing a negatively ionized nanometered far infrared radiating substance, one far infrared radiating warp stitch threads 1215 and 1217 containing a negatively ionized nanometered far infrared radiating substance and multiple weft threads 101. The front spandex warp yarn or filament yarn 1211, the non-stretchable warp yarns 1212, 1214, 1216 and 1218, the far infrared radiating warp threads 1213 and 1219 and the far infrared radiating warp stitch threads 1215 and 1217 are woven with the weft threads 101 subject to the pattern shown in FIG. 4. For example, the non-stretchable warp yarn 1212 extends beneath the first weft thread 101, and then over the top side of the second weft thread 101, and then beneath the third weft thread 101, and then over the top side of the fourth, fifth, sixth, seven and eighth weft threads 101. Subject to this weaving method, each woven zone 12 has a high structural strength.

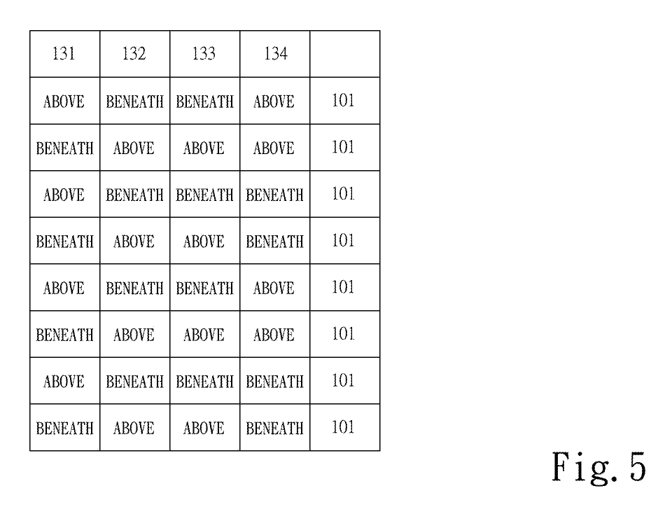

The belt body 1 further comprises a rear selvedge zone 13 jointed to the last woven zone 12. The rear selvedge zone 13 comprises multiple far infrared radiating warp threads 131 and 132 containing a negatively ionized nanometered far infrared radiating substance, multiple spandex warp yarn or filament yarns 133 and 134 and multiple weft threads 101. The far infrared radiating warp threads 131 and 132 and the spandex warp yarn or filament yarns 133 and 134 are woven with the weft threads 101 in a crossed manner. Subject to the pattern shown in FIG. 5. For example, the far infrared radiating warp thread 131 extends over the top side of the first weft thread 101, and then beneath the second weft thread 101, and then over the top side of the third weft thread 101, and then beneath the fourth weft thread 101, and then over the top side of the fifth weft thread 101, and then beneath the sixth weft thread 101, and then over the top side of the seventh weft thread 101 and finally beneath the eighth weft thread 101.

Further, the non-stretchable warp yarns 1212, 1214, 1216 and 1218 of the warp thread units 121 of the woven zones 12 can be prepared from synthetic fiber or natural fiber. The far infrared radiating warp threads 1213 and 1219 of the warp thread units 121 of the woven zones 12 can be prepared from synthetic fiber or natural fiber coated with a negatively ionized nanometered far infrared radiating substance. The far infrared radiating warp stitch threads 1215 and 1217 of the warp thread units 121 of the woven zones 12 can be prepared from synthetic fiber or natural fiber coated with a negatively ionized nanometered far infrared radiating substance. The weft threads 101 can be prepared from synthetic fiber or natural fiber coated with a negatively ionized nanometered far infrared radiating substance.

Further, the far infrared radiating warp threads 113 and 114 of the front edge zone 11 of the belt body 1 can be prepared from synthetic fiber or natural fiber coated with a negatively ionized nanometered far infrared radiating substance.

Further, the far infrared radiating warp threads 131 and 132 of the rear selvedge zone 13 of the belt body 1 can be prepared from synthetic fiber or natural fiber coated with a negatively ionized nanometered far infrared radiating substance.

Further, the tailpiece 3 can be made of plastics through an injection molding process, having a mouth 31 disposed at its one end to facilitate connection to the rear end of the belt body 1.

In conclusion, the invention provides a far infrared waist belt that has the following features and advantages:

1. The far infrared radiating warp threads 1213 and 1219 and far infrared radiating warp stitch threads 1215 and 1217 of the warp thread units 121 of the woven zones 12 as well as the weft threads 101 of the belt body 1 are prepared from synthetic fiber or natural fiber coated with a negatively ionized nanometered far infrared radiating substance. When the far infrared waist belt is fastened to the waist of a person, the far infrared waist belt radiates far infrared rays to stimulate the user's circulation of blood. Further, the negative ions contained in the belt body 1 provide a sterilizing effect.

2. The tailpiece 3 can be directly injection-molded from plastics, having a mouth 31 disposed at its one end to facilitate connection to the rear end of the belt body 1 with stitches.

3. The non-stretchable warp yarns 1212, 1214, 1216 and 1218, far infrared radiating warp stitch threads 1215 and 1217 and far infrared radiating warp threads 1213 and 1219 of the warp thread units 121 are woven with the respective weft threads 101 in a crossed manner, providing a high structural strength.

4. The radiation of far infrared rays from the far infrared radiating substances contained in the belt body 1 penetrates deeply into the user's body to stimulate the circulation of blood and to regulate the functioning of the user's autonomic nervous system. Therefore, the far infrared waist belt can be used as a medical apparatus.

5. The use of the far infrared waist belt is save, and will never cause any allergic to medicine or medical errors.

6. The use of the far infrared waist belt helps the user relieve soreness and pain after exercise or work.

Although a particular embodiment of the invention has been described in detail for purposes of illustration, various modifications and enhancements may be made without departing from the spirit and scope of the invention. Accordingly, the invention is not to be limited except as by the appended claims.

What is claimed is:

1. A far infrared waist belt, comprising a belt body, a buckle fastened to one end of said belt body, and a tailpiece fastened to an opposite end of said belt body, wherein:
said belt body comprises a plurality of woven zones connected one after another, each said woven zone comprising a plurality of weft threads and a plurality of warp thread units woven with the weft threads in a crossed manner, each said warp thread unit comprising a front spandex warp yarn, a plurality of non-stretchable warp yarns, a plurality of far infrared radiating warp threads containing a negatively ionized nanometered far infrared radiating substance and at least one far infrared radiating warp stitch thread containing a negatively ionized nanometered far infrared radiating substance.

2. The far infrared waist belt as claimed in claim 1, wherein said belt body further comprises a front edge zone joined to a front woven zone of said woven zones, said front edge zone comprising a plurality of weft threads and at least one spandex warp yarn and at least one far infrared radiating warp thread containing a negatively ionized nanometered far infrared radiating substance, the at least one spandex warp yarn and at least one far infrared radiating warp thread of said front edge zone being woven with the respective weft threads in a crossed manner.

3. The far infrared waist belt as claimed in claim 2, wherein said front edge zone further comprises a selvedge thread knitted on the weft threads of said front edge zone to form a selvedge.

4. The far infrared waist belt as claimed in claim 1, wherein said belt body further comprises a rear selvedge zone jointed a rear side of said woven zones, said rear selvedge zone comprising a plurality of weft threads, at least one far infrared radiating warp thread containing a negatively ionized nanometered far infrared radiating substance and at least one spandex warp yarn, the at least one far infrared radiating warp thread and at least one spandex warp yarn of said rear selvedge zone being woven with the weft threads of said rear selvedge zone in a crossed manner.

5. The far infrared waist belt as claimed in claim 1, wherein the weft threads, front spandex warp yarn, non-stretchable warp yarns, far infrared radiating warp threads and far infrared radiating warp stitch thread are selectively prepared from synthetic and/or natural fiber and coated with a negatively ionized nanometered far infrared radiating substance.

6. The far infrared waist belt as claimed in claim 2, wherein the weft threads, at least one spandex warp yarn and at least one far infrared radiating warp thread of said front edge zone are selectively prepared from synthetic and/or natural fiber and coated with a negatively ionized nanometered far infrared radiating substance.

7. The far infrared waist belt as claimed in claim 4, wherein the weft threads, at least one far infrared radiating warp thread and at least one spandex warp of said yarn rear selvedge zone are selectively prepared from synthetic and/or natural fiber and coated with a negatively ionized nanometered far infrared radiating substance.

8. The far infrared waist belt as claimed in claim 1, wherein said belt body is stretchable.

9. The far infrared waist belt as claimed in claim 1, wherein said belt body is non-stretchable.
